(19) United States
(12) Patent Application Publication
(10) Pub. No.: US 2020/0000596 A1
(43) Pub. Date: Jan. 2, 2020

(54) PROSTHESIS ADAPTER

(71) Applicant: KONINKLIJKE PHILIPS N.V., EINDHOVEN (NL)

(72) Inventors: SVEN PREVRHAL, HAMBURG (DE); MICHAEL GRASS, BUCHHOLZ IN DER NORDHEIDE (DE); EBERHARD SEBASTIAN HANSIS, Muenchen (DE); KLAUS ERHARD, HAMBURG (DE)

(21) Appl. No.: 16/482,727

(22) PCT Filed: Jan. 30, 2018
(86) PCT No.: PCT/EP2018/052182

§ 371 Date: Aug. 1, 2019

Related U.S. Application Data
(60) Provisional application No. 62/454,130, filed on Feb. 3, 2017.

(51) Int. Cl. A61F 2/30; A61F 2/36; G06F 17/50; G16H 50/50

(52) U.S. Cl. CPC A61F 2/30942; A61F 2/36; A61F 2002/30948; G16H 50/50; A61F 2002/30945; G06F 17/50

(57) ABSTRACT

Patient-specific prosthetic apparatus and methods. The patient-specific prosthetic apparatus includes a prosthetic device and a prosthetic adapter configured to be secured with the prosthetic device, wherein the prosthetic adapter includes an interior surface that matches the shape of a portion of the prosthetic device, and an exterior surface that matches a patient's anatomy.

### TECHNICAL FIELD

Various embodiments described herein relate to methods and devices directed to prosthetics and, more particularly but not exclusively, to methods and devices related to patient-specific prosthetics.

### BACKGROUND

High-fidelity three-dimensional (3D) imaging techniques such as computerized tomography (CT) scanning, magnetic resonance imaging (MRI), and image post-processing have been useful in generating prosthetic devices. These prosthetic devices may include devices for hips, arms, legs, shoulders, skull caps, devices associated with soft tissue (e.g., tracheal splints), or the like.

3D printed components such as skull plates, splints, dental implants, and functional tissues are now widely available. 3D printed components may become even more prevalent in healthcare as the cost of printing decreases, the speed of printing increases, and the operation of printers becomes easier. As this occurs, 3D printing may become a standard tool in applications such as planning operations, simulating surgical tasks, developing implants, and guiding operations.

However, 3D printing is not without its shortcomings. For one, mechanical and functional needs vary considerably amongst different patients. Accordingly, a given 3D-printed stock material may be satisfactory for one patient but not another patient. Improper matches between a prosthetic device and a patient may result in an unacceptable load on a particular material or host tissue and result in undesired adaptive tissue changes. This in turn may lead to failure of the prosthetic device at the host-implant interface or even failure of the prosthetic device itself.

Another disadvantage of existing 3D printing procedures is the high level of staff training and resources needed to acquire image(s), generate templates, and print the final device.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to the foregoing, it would be desirable to provide methods and apparatus that combine mass manufactured prosthetic devices with patient-specific prosthetic adapters that are easily manufactured and can satisfy the functional and mechanical requirements of a particular patient.

In one aspect, various embodiments relate to a method for generating a patient-specific prosthetic apparatus. The method includes receiving a three-dimensional model of a patient's anatomy; receiving a three-dimensional template of a prosthetic device; subtracting a portion of the three-dimensional template of the prosthetic device from the three-dimensional model of the patient's anatomy to generate a differential model for a prosthetic adapter; and manufacturing a three-dimensional prosthetic adapter based on the differential model for the prosthetic adapter, wherein the manufactured prosthetic adapter includes an interior surface that matches the shape of the portion of the prosthetic device, and an exterior surface that matches the patient's anatomy.

In one embodiment, the method further includes securing the prosthetic device to the manufactured prosthetic adapter. In one embodiment, securing the prosthetic device to the prosthetic adapter includes securing the prosthetic device at least partially within the prosthetic adapter.

In one embodiment, the prosthetic device is secured to the prosthetic adapter via a plurality of threads on each of the prosthetic device and the prosthetic adapter or via at least one pin.

In one embodiment, the method further includes gathering imagery of the patient's anatomy. In one embodiment, the method further includes converting the gathered imagery of the patient's anatomy to the three-dimensional model of the patient's anatomy.

In one embodiment, the prosthetic adapter is manufactured by three-dimensional printing.

In one embodiment, the prosthetic adapter is configured to accommodate locally varying rigidity and elasticity requirements.

In another aspect, various embodiments relate to a patient-specific prosthetic apparatus. The apparatus includes a prosthetic device; and a prosthetic adapter configured to be secured with the prosthetic device, wherein the prosthetic adapter includes: an interior surface that matches the shape of a portion of the prosthetic device, and an exterior surface that matches a patient's anatomy.

In one embodiment, the prosthetic device is secured at least partially within the prosthetic adapter.

In one embodiment, the prosthetic adapter is manufactured by three-dimensional printing.

In one embodiment, the prosthetic adapter is configured to accommodate locally varying rigidity and elasticity requirements.

In one embodiment, the prosthetic device is secured to the prosthetic adapter via a plurality of threads on each of the prosthetic device and the prosthetic adapter or via at least one pin.

In one embodiment, the prosthetic adapter is configured as a plurality of components.

In yet another aspect, various embodiments relate to a computer readable medium containing computer-executable instructions for generating a patient-specific prosthetic apparatus. The medium includes computer-executable instructions for receiving a three-dimensional model of a patient's anatomy; computer-executable instructions for receiving a three-dimensional template of a prosthetic device; computer-executable instructions for subtracting a portion of the three-dimensional template of the prosthetic device from the three-dimensional model of the patient's anatomy to generate a differential model for a prosthetic adapter; and computer-executable instructions for manufacturing a three-dimensional prosthetic adapter based on the differential model for the prosthetic adapter, wherein the manufactured prosthetic adapter includes: an interior surface that matches the shape of the portion of the prosthetic device, and an exterior surface that matches the patient's anatomy.

Various embodiments described herein relate to method for generating a patient-specific prosthetic apparatus, a device including a processor for performing the method, and a computer-readable medium encoded with instructions for causing a processor to perform the method; the method including: obtaining image data representing a portion of a patient's anatomy; obtaining a three-dimensional stock template representing at least a portion of a stock device; generating a three-dimensional adapter model representing an adapter device based on the image data and the stock template, wherein the adapter model defines at least one cavity that is complementary to the portion of the stock device and a surface defined by the image data.

Various embodiments are described wherein: the image data includes three-dimensional image data; and generating the three-dimensional adapter model includes subtracting volume data of the stock template from volume data of the image data.

Various embodiments are described wherein: the image data represents bone tissue as a three-dimensional volume and a medullary cavity of the patient as empty space; and generating the three-dimensional adapter model includes: aligning the image data and stock template such that the portion of the stock device is disposed within the medullary cavity, and generating volume data representative of volume between the portion of the stock device and the bone tissue.

Various embodiments are described wherein the stock template defines at least one linkage structure on the portion of the stock device, whereby generating the three-dimensional adapter model generates at least one complementary linkage structure on the adapter device.

Various embodiments are described wherein obtaining image data representing a portion of the patient's anatomy includes receiving a three dimensional anatomy model obtained by an imaging device.

Various embodiments are described wherein obtaining image data representing a portion of the patient's anatomy includes: receiving medical scan data obtained by an imaging device; and converting the medical scan data to a three-dimensional anatomy model.

Various embodiments additionally include transmitting the three-dimensional adapter model to a three-dimensional printer for production.

Various embodiments are described wherein the three-dimensional adapter model includes material metadata that is configured to accommodate locally varying rigidity and elasticity requirements

### BRIEF DESCRIPTION OF DRAWINGS

In order to better understand various exemplary embodiments, reference is made to the accompanying drawings, wherein:

FIG. 1 illustrates a system for generating a patient-specific prosthetic apparatus in accordance with one embodiment;

FIG. 2 illustrates a model of a portion of a patient's anatomy in accordance with one embodiment;

FIG. 3 illustrates a template of a stock prosthetic device in accordance with one embodiment;

FIG. 4 illustrates a front view of the template of FIG. 3 in accordance with one embodiment;

FIG. 5 illustrates a side view of the template of FIG. 3 in accordance with one embodiment;

FIGS. 6A-B illustrate portions of the template of FIG. 3 overlaid onto the model of FIG. 2;

FIG. 7 illustrates a differential model for a prosthetic adapter in accordance with one embodiment;

FIG. 8 illustrates a manufactured prosthetic adapter in accordance with one embodiment;

FIG. 9 illustrates a patient-specific prosthetic apparatus being assembled in accordance with one embodiment;

FIG. 10 illustrates an assembled patient-specific prosthetic apparatus in accordance with one embodiment;

FIG. 11 illustrates a prosthetic device and a prosthetic adapter with threads in accordance with one embodiment;

FIGS. 12A-B illustrate a prosthetic device and a prosthetic adapter with a pin and latch security mechanism in accordance with one embodiment;

FIG. 13 depicts a flowchart of a method of generating a patient-specific prosthetic apparatus in accordance with one embodiment;

FIG. 14 depicts a flowchart of a method of generating a patient-specific prosthetic apparatus in accordance with another embodiment; and

FIG. 15 illustrates an example of a hardware device for implementing the methods described herein in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, which may encompass both volatile and non-volatile memories (but exclude transitory signals), such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

In view of the disadvantages of existing prosthetic devices and methods discussed above, features of various embodiments described herein relate to a patient-specific prosthetic apparatus that includes a stock prosthetic device and a prosthetic adapter. This apparatus and methods described herein combine stock prosthetic devices that are mass-manufactured to precisely match desired functional properties (e.g., mechanical stiffness) with a 3D-printed prosthetic adapter. The prosthetic adapter essentially serves as a “jacket” for the prosthetic device. The prosthetic adapter may provide the desired patient-specific anatomical shape, while also tolerating looser requirements in terms of its functional properties.

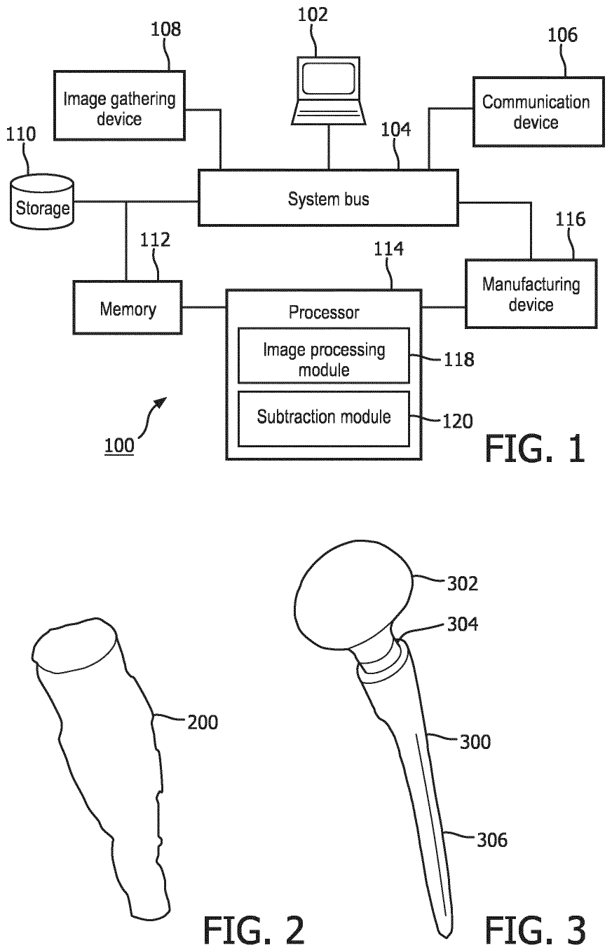

FIG. 1 illustrates an exemplary system 100 for generating a patient-specific prosthetic apparatus in accordance with one embodiment. The system 100 may include a user interface 102 accessible by an operator, a system bus 104, a communication device 106, an image gathering device 108, a storage 110, memory 112, a processor 114, and a manufacturing device 116. Various alternative components or arrangements thereof suitable for practicing the principles described herein will be apparent. For example, it will be understood that, in some embodiments, one or more of the image gathering device 108 and manufacturing device 116 may be physically separate and independent devices from a device incorporating the processor 114 (and other components of the system 100). Further, in some embodiments, certain components may be omitted entirely. For example, in some embodiments, the user interface 102 may be omitted and the processor 114 may be instructed to drive creation of an implant by an instruction message received from another device via the communication device 106.

The user interface 102 may be any sort of interface that can receive instructions from an operator (e.g., medical personnel) and present information to an operator. The user interface 102 may include a display, speaker, microphones, and may be configured as a PC, laptop, tablet, mobile device, or the like. The exact configuration of the user interface(s) 102 may vary as long as the user interface(s) 102 can receive instructions from an operator and present information to an operator.

The system bus 104 may enable information to be transferred among the various devices of the system 100. For example, the system bus may enable instructions to be communicated from the user interface 102 to the image gathering device 108.

The communication device 106 may enable communication between the components of the system 100 and other external components. For example, the communication device 106 may include a wired or wireless network interface, USB interface, or other interface for allowing messages to be exchanged between devices. The communication device 106 may, for example, receive templates, models, or other types of imagery related to one or more patients. Similarly, the communication device 106 may communicate data (e.g., imagery) regarding a particular patient to other devices external to the system 100. This communication may be made by any type of wired or wireless connections known in the art.

The image gathering device 108 may be any type of imaging device that can gather information regarding a patient's anatomy. The image gathering device 108 may be a device that can gather 2D or 3D images of a patient's anatomy (or a portion of the patient's anatomy). In one embodiment the image gathering device 108 may be a computer tomography (CT) scanning device. In these embodiments, the CT scanning device may gather and combine a plurality of X-ray images taken from various positions about a patient. These plurality of images may be combined to create, for example, cross-sectional images or slices of various portions of the patient's anatomy. In other embodiments, the image gathering device 108 may be a magnetic resonance imaging (MRI) device. In some embodiments, the image gathering device 108 may not communicate directly with the system bus 104 and, instead, may be a physically separate device and may communicate with, e.g., the processor 114 via the communications device 106.

The image gathering device 108 need not be a 3D imaging device. In some embodiments, the image gathering device 108 may gather two dimensional (2D) imagery of a patient's anatomy. For example, the image gathering device 108 may gather a plurality of 2D X-ray images that can be used to form 3D model(s) using projection techniques known in the art. In some embodiments, 2D images may be used to modify a pre-existing template 3D model such as, for example, where acquired 2D images along are not sufficient to reconstruct a full 3D model. In such an embodiment, a 2D image may at least be sufficient to customize one or more surface portions of a template 3D model based on the anatomical profile(s) that can be observed. In some such embodiments, subsequent interpolation of other surfaces on the 3D model that are not visible in the available images may, in some applications, provide a useful ‘partial’ customization of the implant.

The storage 110 may store various types of data in a variety of formats. For example, the storage 110 may store data related to a particular patient such as their anatomy, as well as other types of medical information.

The storage 110 may also store any number of 3D templates of stock prosthetic devices. These prosthetic devices may be previously modeled and may be available through public and/or private services.

The storage 110 may store 3D templates of prosthetic devices for a variety of different body parts. These may include models of prosthetic hip devices, prosthetic arm devices, prosthetic leg devices, or the like. There may also be a wide variety of shapes and sizes for these various prosthetic devices to accommodate patients of various sizes, builds, and shapes. For example, the storage 110 may store a plurality of templates of femur prosthetic devices that may vary in length and thickness. While various embodiments described herein relate to femoral intramedullary implants, modifications for application of the principles to other forms of implants (e.g., intramedullary or otherwise) will be apparent.

The memory 112 may be L1, L2, L3 cache or RAM memory configurations. The memory 112 may include non-volatile memory such as flash memory, EPROM, EEPROM, ROM, and PROM, or volatile memory such as static or dynamic RAM, as discussed above. The exact configuration/type of memory 112 may of course vary as long as information such as instructions related to model generation, template generation, and manufacturing can be stored and retrieved.

The processor 114 may be any hardware device capable of executing instructions stored in memory 112 to process data regarding imagery, to generate models, and to provide instructions to the manufacturing device 116. The processor 114 may be a microprocessor, a field programmable gate array (FPGA), an application-specific integrated circuit (ASIC), or a similar type of device. In some embodiments, such as those relying on one or more ASICs, the functionality described as being provided in part via software may instead be configured into the design of the ASICs and, as such, the associated software may be omitted.

The processor 114 may further include an image processing module 118 and a subtraction module 120. The image processing module 118 may perform any necessary processing steps on the received imagery. For example, the image processing module 118 may process a plurality of X-ray images of the patient's anatomy to generate a 3D model of the patient's anatomy.

The processor 114 may also query the storage 110 to retrieve one or more previously stored 3D prosthetic device templates. Or, an orthopedic surgeon or other medical personnel may query the storage 110 via the user interface 102 for an appropriate template from a set of stock prosthetic device templates. The orthopedic surgeon or the like may select this template based on any number of criteria.

FIG. 2 illustrates an exemplary 3D model 200 of a patient's anatomy. As illustrated, this model 200 is a 3D model of the femoral portion of the patient. However, the features of various embodiments described herein may be directed towards any portion of a patient's anatomy.

FIG. 3 illustrates a 3D template 300 of a femur prosthetic device. The template 300 may include a head portion 302, a neck portion 304, and a shaft portion 306. This particular prosthetic device (and its corresponding template 300) may have been chosen based on in its similarity in size to the 3D model 200 of FIG. 2 and/or based on any number of criterion. The actual prosthetic device which the template 300 is based on may be readily accessible by an operator such as medical personnel. For example, the particular healthcare institution may have large quantities of various stock prosthetic devices. These prosthetic devices are generally formed from steel or titanium.

The 3D model 200 of the patient's anatomy and the 3D template 300 of the stock prosthetic device may then be communicated to the subtraction module 120. The model 200 and the template 300 may be presented to an operator such an orthopedic surgeon via a display on the user interface 102 using any suitable computer aided drawing (CAD) tool. The model 200 and the template 300 may be positioned in the same coordinate system of any type of CAD tool.

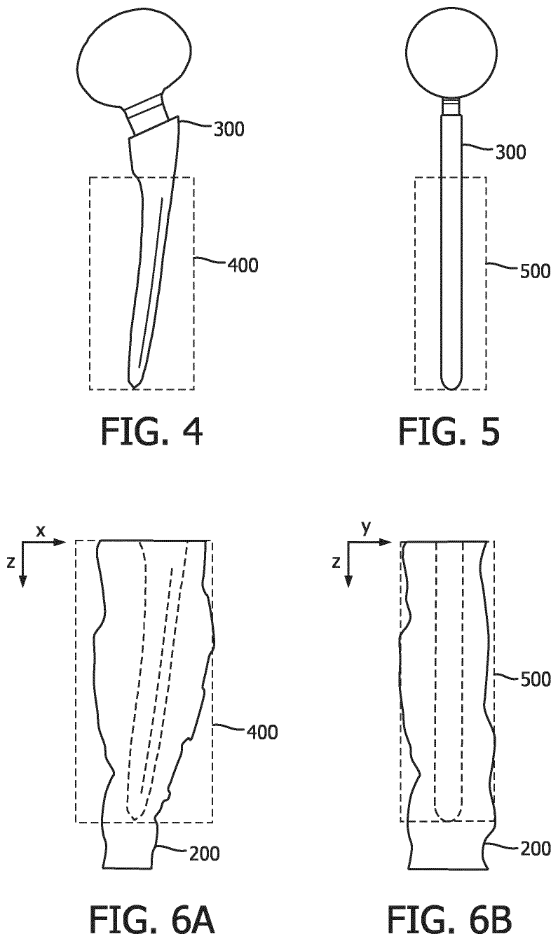

Next, a portion of the template 300 may be selected. For example, FIG. 4 illustrates front view of the template 300 and a selected portion 400 of the template 300. This selected portion 400 may be defined by an operator such as medical personnel or the like. The operator may select this portion 400 by, for example, dragging a cursor over the portion 400. Although the portion 400 is defined in two dimensions, the highlighted portion 400 may also be defined in three dimensions. FIG. 5 illustrates a side view of the template 300 and similarly illustrates another selected portion 500 of the template 300.

The selected portions 400 and 500 (as well as any other required selected portions) may then be overlaid onto the model 200. For example, the highlighted portion 400 and the model 200 may be positioned in the same coordinate system in a CAD program. The operator may position the selected portion 400 over the model 200 such that the top of the model 200 and the top of the selected portion 400 line up with each other as illustrated in FIG. 6A. Accordingly, the selected portion 400 of the template 300 is “within” the model 200.

Similarly, FIG. 6B illustrate the selected portion 500 overlaid on a side view of the model 200 (indicated by the set of axes). As in FIG. 6A, the operator may position the selected portion 500 such that the top of the selected portion 500 lines up with the top of the model 200. Accordingly, the portion 500 of the template 300 is “within” the model 200.

The subtraction module 120 may then subtract or otherwise remove a portion of the model 200 that corresponds to the shape and size of the portion of the template selected in FIGS. 4 and 5. The subtraction module 120 may subtract this portion using any suitable CAD techniques, commands, or tools. It will be apparent that, while the term ‘subtraction’ is used in describing the various embodiments herein, other operations may be appropriate in other embodiments depending on the form of the image data or model data. For example, subtraction may be appropriate for an intramedullary implant where the anatomical model 200 represents an intramedullary cavity as a solid volume. In an alternative embodiment, wherein the solid volume of the anatomical model 200 represents bone or other tissue and the intramedullary cavity is represented as the absence of volume data, the models 200, 300 may be aligned and an inversion operation may be performed to create a model of the adapter.

It will also be apparent that subtraction, inversion, or other operations between the two models 200, 300 may in some embodiments not be the final operation before the model is ready to print. For example, in some embodiments (e.g., those using an inversion), extra volume such as any volume entirely outside the bone may be “clipped” from the inverted model. In some embodiments, the surfaces of the adapter model may be slightly shrunk or expanded with respect to a central axis to provide for a wider tolerance fit between the adapter and the stock implant or patient anatomy. In some embodiments, additional subtractions may be performed to ensure that the adapter can be fully inserted longitudinally into the patient's medullary cavity (e.g., internal prominences may be smoothed). Various additional post-processing steps will be apparent.

In some embodiments, subtraction may be performed without any manual selections. For example, the two models 200, 300 may be automatically aligned based on common reference points (e.g., metadata in both indicating an origin or other point of interest). Alternatively, the processor may simply align the implant model within a cavity defined in the image data (e.g., a medullary cavity) at one of many possible alignments accomplishing disposition within the cavity. In some embodiments, the implant model 300 may only depict a portion of the physical stock implant to be used such as, for example, only those portions to be inserted into bone or to be disposed within the adapter to be created.

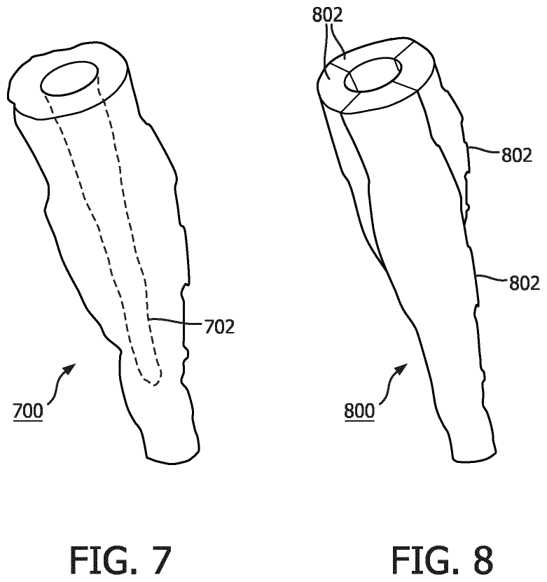

FIG. 7 illustrates an isometric view of a differential model 700 for a prosthetic adapter (which is the model 200 after the subtraction). As can be seen, a portion 702 has been removed (indicated by the dashed lines). The removed portion 702 has at least substantially the same shape and size of the portions of the template 300 that were selected in FIGS. 4 and 5. An operator such as medical personnel, an orthopedic surgeon, or the like may also have the opportunity to perform further modifications to the differential model 700.

The differential model 700 for the prosthetic adapter may be communicated to the manufacturing device 116 (i.e., a computing unit of the manufacturing device 116) to be manufactured. The differential model 700 may be communicated in the form of digital model data, a 3D model, an additive manufacturing file (AMF), a stereolithography (STL) file, or the like. Accordingly, the manufacturing device 116 may be any type of 3D printing or additive manufacturing device. In various embodiments, the manufacturing device 116 may be an independent device and may instead communicate with, e.g., the processor 114 via the communication device 106.

The exact manufacturing process used may vary. The manufacturing device 116 may manufacture the prosthetic adapter via any one of selective laser melting techniques, direct metal laser sintering techniques, selective laser sintering techniques, fused deposition modeling techniques, fused filament fabrication techniques, stereolithography techniques, laminated object manufacturing techniques, or the like. The type of manufacturing technique (as well as the type of manufacturing device 116) may vary, and may depend on speed, costs, and the particular needs of the operator and/or patient.

The material of the manufactured prosthetic adapter may vary as well. Generally, though, the prosthetic adapter may be formed from any type of material whether available now or invented hereafter as long as it can achieve the bio-functional requirements of the particular patient.

The prosthetic adapter need not be modeled/manufactured as one piece. For example, FIG. 8 illustrates a manufactured prosthetic adapter 800 in accordance with one embodiment. In this embodiment, the prosthetic adapter 800 includes four components 802. The components may be joined together via an adhesive substance or mechanical linkages, for example. In some such embodiments, the image processing module 118 may be further adapted to add or subtract some mechanisms providing such linkages to the model 200 created from the patient's anatomical data. In some embodiments, the linkage may be already formed in the stock implant model (e.g., in the case of threads) and the initial subtraction step may already be sufficient to accomplish creation of the complementary linking structure in the adapter model.

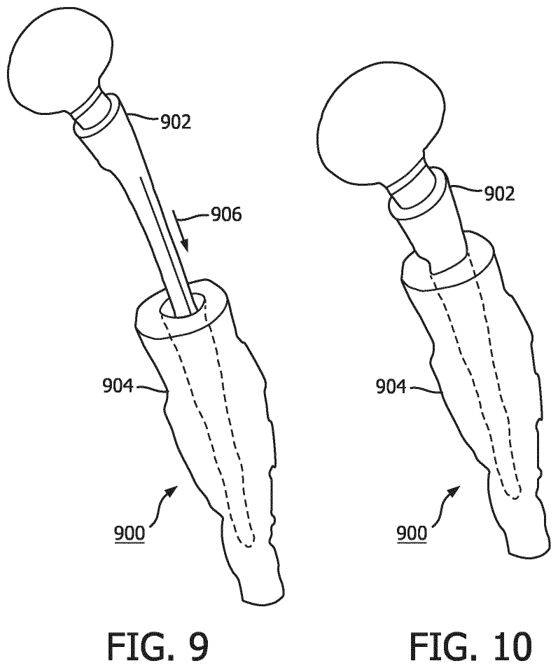

FIG. 9 illustrate a patient specific prosthetic apparatus 900 being assembled in accordance with one embodiment. In FIG. 9, the prosthetic apparatus 900 includes the prosthetic device 902 and a manufactured prosthetic adapter 904 that is based on the differential model 700 of FIG. 7. In this particular embodiment, the prosthetic device 902 is the device that the template 300 of FIG. 3 is based on.

Specifically, FIG. 9 illustrates the prosthetic device 902 being inserted into the prosthetic adapter 904. This insertion is indicated by arrow 906. FIG. 10 illustrates the prosthetic device 902 inserted at least partially into the prosthetic adapter 904.

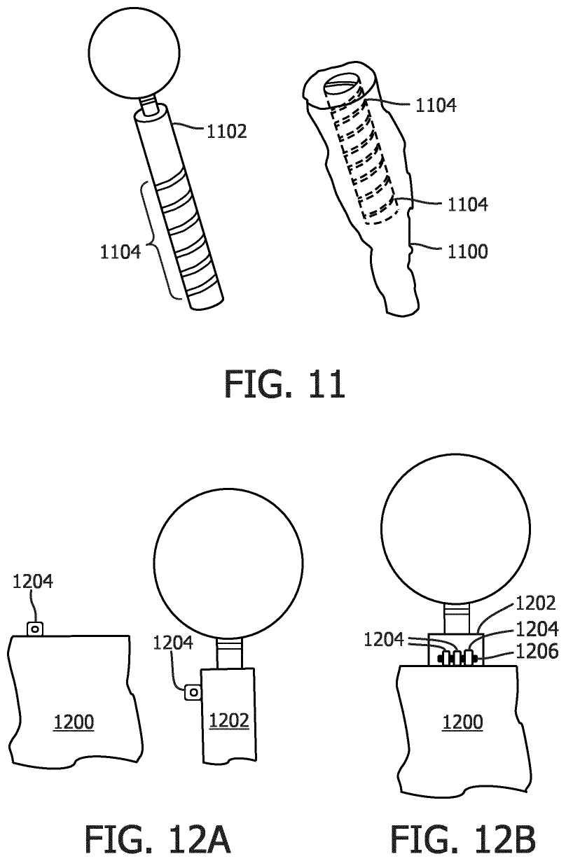

The components of the patient-specific prosthetic apparatus 900 may be fixed or otherwise secured together in a variety of ways. For example, FIG. 11 illustrates a prosthetic adapter 1100 and a prosthetic device 1102 that both include a plurality of threads 1104. Accordingly, the prosthetic device 1102 may be screwed into and secured with the prosthetic adapter 1100 via the plurality of threads 1104.

In other embodiments, the components of the patient-specific prosthetic apparatus may be fixed or otherwise secured together via pin and latch mechanisms. For example, FIG. 12A illustrates partial views of a prosthetic adapter 1200 and a prosthetic device 1202. Each of the prosthetic adapter 1200 and the prosthetic device 1202 may include one or more latch mechanisms 1204 affixed thereon such that when the prosthetic device 1202 is inserted into the prosthetic adapter 1200, the holes of latch mechanisms 1204 concentrically align with each other.

FIG. 12B illustrates the prosthetic device 1202 inserted into the prosthetic adapter 1200. The holes of the latch mechanisms 1204 are aligned such that a pin 1206 is able to be inserted through the holes of each latch mechanism 1204, thereby securing the prosthetic device 1202 with the prosthetic adapter 1200. The pin 1206 may further include removable caps to ensure the pin does not slide out from the latch mechanisms 1204.

These types of mechanisms are merely exemplary, and other securing or fixing methods may similarly be used. For example, the interior of the prosthetic adapter and/or the exterior of the prosthetic device may be coated with an adhesive substance before insertion. Or, the components may be secured purely by a press fit. Once constructed, the patient-specific prosthetic apparatus may be implanted in the patient.

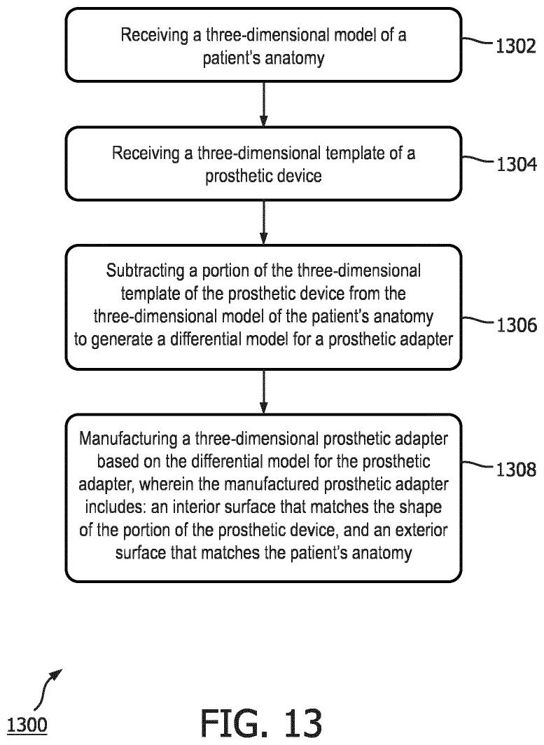

FIG. 13 depicts a flowchart of a method 1300 for generating a patient-specific prosthetic apparatus in accordance with one embodiment. In some embodiments, the method 1300 (or some steps thereof) may correspond to software instructions belonging to the image processing module 118 or subtraction module 120. Step 1302 involves receiving a three-dimensional model of a patient's anatomy. This model may be similar to the model 200 of FIG. 2. The model may be received from any suitable type of image capturing device and/or image processing module. This model essentially serves as the foundation of the patient-specific adapter. The exterior surface of the model may match a portion of the patient's anatomy, such as the shape of a patient's femur (e.g., the internal surface of a patient's femoral medullary cavity). The model may be presented to an operator such as an orthopedic surgeon or other type of interested personnel via a user interface.

Step 1304 involves receiving a three-dimensional template of a prosthetic device. The operator may search or query one or more databases such as the storage 110 for the three-dimensional template. The operator may search for a template of a particular prosthetic device based on the type of prosthetic device, and/or based on the size and shape of the anatomy model received in step 1302. For example, an operator may query the storage 110 via the user interface 102 by inputting or selecting “femoral prosthetic devices” and then filter results based on size, material, etc.

A healthcare institution such as where the operator works may have a stocked supply of a plurality of prosthetic devices, and the storage 110 may store templates of the prosthetic devices that are in stock. Additionally, the storage 110 may store templates of prosthetic devices that are not stocked in the healthcare institution. In this case, the operator may have to order a particular prosthetic device from a supplier.

Step 1306 involves subtracting a portion of the three-dimensional template of the prosthetic device from the three-dimensional model of the patient's anatomy to generate a differential model for a prosthetic adapter. This step may be performed by any suitable CAD program, tool, or command, and may be performed via the methods discussed previously. For example, an operator may select a particular portion of the prosthetic device template that corresponds to a portion to be removed from the model of the patient's anatomy. The size and shape of this portion may vary, as long as the manufactured prosthetic adapter and the prosthetic device can be fixed together to accomplish the various features described herein.

Step 1308 involves manufacturing a three-dimensional prosthetic adapter based on the differential model for the prosthetic adapter, wherein the manufactured prosthetic adapter includes an interior surface that matches the shape of the portion of the prosthetic device, and an exterior surface that matches the patient's anatomy. Step 1308 may be performed by any suitable three-dimensional printing device as discussed previously. The interior surface may match the shape of the portion of the prosthetic device such that the prosthetic adapter can receive at least the portion of the prosthetic device. Additionally, the exterior surface of the prosthetic adapter may at least substantially match the shape of a portion of the patient's anatomy (e.g., the patient's femoral anatomy).

The prosthetic adapter may also be manufactured to accommodate locally varying rigidity and elasticity requirements. These requirements may be based on the particular shape and size of the prosthetic device, the shape and size of the prosthetic adapter, the anatomic portion of interest, as well as other requirements specific to the patient. For example, metadata included with the model may specify that certain portions of the prosthetic adapter may be manufactured to be denser if it will be subject to a high amount of pressure or force. Or, metadata may define materials or properties thereof such that certain portions of the prosthetic adapter may be configured to be more elastic to allow for a certain degree of flexibility.

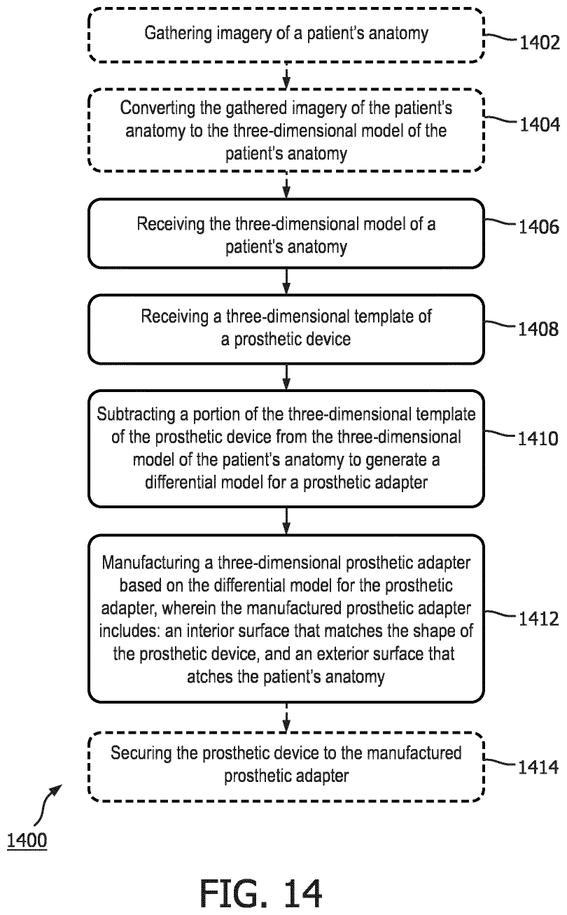

FIG. 14 depicts a flowchart of a method 1400 for generating a patient-specific prosthetic apparatus in accordance with another embodiment. In some embodiments, the method 1400 (or some steps thereof) may correspond to software instructions belonging to the image processing module 118 or subtraction module 120. Steps 1406, 1408, 1410, and 1412 are similar to steps 1302, 1304, 1306, and 1308, respectively, of FIG. 13 and are not repeated here.

Steps 1402, 1404, and 1414 are optional. Steps 1402 involves gathering imagery of a patient's anatomy. This imagery may be of a particular portion of a patient's anatomy, such as the patient's femur or a portion thereof. For example, in the embodiment of the illustrated implants, the imagery may at least capture a proximal portion of the patient's left or right (depending on the intended location of the implant) femoral medullary cavity. This imagery may be gathered at a healthcare institution using, for example, a CT scanner, MRI scanner, or an X-ray machine.

Step 1404 involves converting the gathered imagery of the patient's anatomy into a three-dimensional model of the patient's anatomy. The gathered imagery may be converted to a 3D model using any suitable image analysis tool, program, technique, or command.

Step 1414 may occur after the prosthetic adapter is manufactured, and involves securing the prosthetic device to the manufactured prosthetic adapter. As mentioned previously, the operator may have access to a plurality of stock prosthetic devices. The operator may therefore obtain an actual prosthetic device that the prosthetic device template is based on. The prosthetic device and the prosthetic adapter may be secured to each other in a variety of ways. As discussed previously, these may include threads, pin and latch mechanisms, adhesives, or press fits. The exact device or technique used may vary as long as the prosthetic device and the prosthetic adapter can be secured together to accomplish the various features described herein.

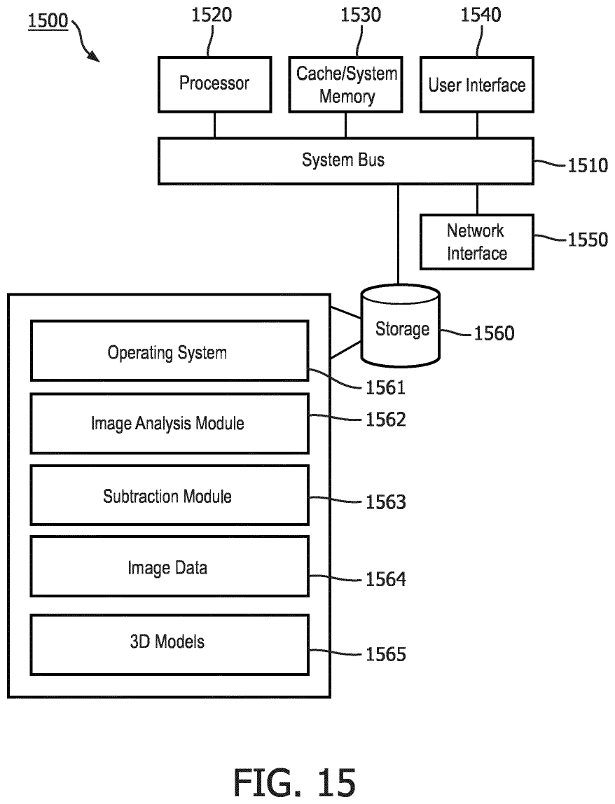

FIG. 15 illustrates an exemplary hardware device 1500 for generating a patient-specific prosthetic apparatus in accordance with one embodiment. As shown, the device 1500 includes a processor 1520, memory 1530, user interface 1540, network interface 1550, and storage 1560 interconnected via one or more system buses 1510. It will be understood that FIG. 15 constitutes, in some respects, a necessary simplification and that the actual organization of the components of the device 1500 may be more complex than illustrated.

The processor 1520 may be any hardware device capable of executing instructions stored in memory 1530 or storage 1560 or otherwise capable of processing data. As such, the processor may include a microprocessor, field programmable gate array (FPGA), application-specific integrated circuit (ASIC), or other similar devices.

The memory 1530 may include various memories such as, for example L1, L2, or L3 cache or system memory. As such, the memory 1530 may include static random access memory (SRAM), dynamic RAM (DRAM), flash memory, read only memory (ROM), or other similar memory devices.

The user interface 1540 may include one or more devices for enabling communication with a user. For example, the user interface 1540 may include a display, a mouse, and a keyboard for receiving user commands. In some embodiments, the user interface 1540 may include a command line interface or graphical user interface that may be presented to a remote terminal via the network interface 1550.

The network interface 1550 may include one or more devices for enabling communication with other hardware devices. For example, the network interface 1550 may include a network interface card (NIC) configured to communicate according to the Ethernet protocol. Additionally, the network interface 1550 may implement a TCP/IP stack for communication according to the TCP/IP protocols. Various alternative or additional hardware or configurations for the network interface 1550 will be apparent.

The storage 1560 may include one or more machine-readable storage media such as read-only memory (ROM), random-access memory (RAM), magnetic disk storage media, optical storage media, flash-memory devices, or similar storage media. In various embodiments, the storage 1560 may store instructions for execution by the processor 1520 or data upon with the processor 1520 may operate.

For example the storage 1560 may include an operating system 1561 that includes an image analysis module 1562 for processing gathered imagery of a patient's anatomy and converting it to a 3D model, a subtraction module 1663 for subtracting a portion of a stock prosthetic device template from the 3D model to form a differential model for a prosthetic adapter, image data 1564 (e.g., data retrieved from an imaging device for a patient) for customizing an adapter, and 3D models of stock implants, adapter implants, etc.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for generating a patient-specific prosthetic apparatus, the method comprising:
obtaining image data representing a portion of a patient's anatomy;
obtaining a three-dimensional stock template representing at least a portion of a stock device;
generating a three-dimensional adapter model representing an adapter device based on the image data and the stock template, wherein the adapter model defines at least one cavity that is complementary to the portion of the stock device and a surface defined by the image data.

2. The method of claim 1, wherein:
the image data comprises three-dimensional image data; and
generating the three-dimensional adapter model comprises subtracting volume data of the stock template from volume data of the image data.

3. The method of claim 1, wherein:
the image data represents bone tissue as a three-dimensional volume and a medullary cavity of the patient as empty space; and
generating the three-dimensional adapter model comprises:
aligning the image data and stock template such that the portion of the stock device is disposed within the medullary cavity, and
generating volume data representative of volume between the portion of the stock device and the bone tissue.

4. The method of claim 1, wherein the stock template defines at least one linkage structure on the portion of the stock device,
whereby generating the three-dimensional adapter model generates at least one complementary linkage structure on the adapter device.

5. The method of claim 1, wherein obtaining image data representing a portion of the patient's anatomy comprises receiving a three dimensional anatomy model obtained by an imaging device.

6. The method of claim 1, wherein obtaining image data representing a portion of the patient's anatomy comprises:
receiving medical scan data obtained by an imaging device, and
converting the medical scan data to a three-dimensional anatomy model.

7. The method of claim 1, further comprising transmitting the three-dimensional adapter model to a three-dimensional printer for production.

8. The method of claim 1, wherein the three-dimensional adapter model includes material metadata that is configured to accommodate locally varying rigidity and elasticity requirements.

9. A computer readable medium containing computer-executable instructions for generating a patient-specific prosthetic apparatus, the medium comprising:
instructions for obtaining image data representing a portion of a patient's anatomy;
instructions for obtaining a three-dimensional stock template representing at least a portion of a stock device;
instructions for generating a three-dimensional adapter model representing an adapter device based on the image data and the stock template, wherein the adapter model defines at least one cavity that is complementary to the portion of the stock device and a surface defined by the image data.

10. The computer readable medium of claim 9, wherein:
the image data comprises three-dimensional image data; and
generating the three-dimensional adapter model comprises subtracting volume data of the stock template from volume data of the image data.

11. The computer readable medium of claim 9, wherein:
the image data represents bone tissue as a three-dimensional volume and a medullary cavity of the patient as empty space; and
the instructions for generating the three-dimensional adapter model comprise:
instructions for aligning the image data and stock template such that the portion of the stock device is disposed within the medullary cavity, and
instructions for generating volume data representative of volume between the portion of the stock device and the bone tissue.

12. The computer readable medium of claim 1, wherein the stock template defines at least one linkage structure on the portion of the stock device,
whereby the instructions for generating the three-dimensional adapter model generate at least one complementary linkage structure on the adapter device.

13. The computer readable medium of claim 1, wherein the instructions for obtaining image data representing a portion of the patient's anatomy comprise instructions for receiving a three dimensional anatomy model obtained by an imaging device.

14. The computer readable medium of claim 1, wherein the instructions for obtaining image data representing a portion of the patient's anatomy comprise:
instructions for receiving medical scan data obtained by an imaging device
instructions for converting the medical scan data to a three-dimensional anatomy model.

15. The computer readable medium of claim 1, further comprising instructions for transmitting the three-dimensional adapter model to a three-dimensional printer for production.
